(19) United States
(12) Patent Application Publication
(10) Pub. No.: US 2007/0276050 A1
(43) Pub. Date: Nov. 29, 2007

(54) Methods for identifying ASK1 inhibitors useful for preventing and/or treating cardiovascular diseases

(75) Inventors: Keith A. Koch, Erie, CO (US); Lawrence S. Melvin JR., Longmont, CO (US); Richard J. Gorczynski, Westminster, CO (US)

Correspondence Address: HARNESS, DICKEY, & PIERCE, P.L.C, 7700 BONHOMME, STE 400, ST. LOUIS, MO, 63105, US

(73) Assignee: GILEAD SCIENCES, INC., Foster City, CA (US)

(21) Appl. No.: 11/711,315

(22) Filed: Feb. 26, 2007

Related U.S. Application Data
(60) Provisional application No. 60/777,390, filed on Feb. 27, 2006.

(51) Int. Cl. C12Q 1/48; A61K 31/00; A61P 9/00

(52) U.S. Cl. 514/789; 435/15

(57) ABSTRACT

This invention is directed to methods for identifying apoptosis signal-regulated kinase 1 (“ASK1”) inhibitors useful for preventing and/or treating cardiovascular disease. This invention also relates to methods for preventing and/or treating cardiovascular disease in an animal by administering to the animal an ASK1 inhibitor.

### PRIORITY CLAIM TO RELATED PATENT APPLICATIONS

This patent claims priority to U.S. Provisional Patent Application No. 60/777,390 (filed Feb. 27, 2006). The entire text of the '390 application is incorporated by reference into this patent

### FIELD OF THE INVENTION

This invention is directed to methods for identifying apoptosis signal-regulated kinase 1 (“ASK1”) inhibitors useful for preventing and/or treating cardiovascular disease. This invention also related to methods for preventing and/or treating cardiovascular diseases in animals by administering to the animals an ASK1 inhibitor.

### BACKGROUND OF THE INVENTION

ASK1 is a member of the mitogen-activated protein kinase kinase kinase (“MAP3K”) family that activates the c-Jun N-terminal protein kinase (“JNK”) and p38 MAP kinase. Members of the MAP3K kinase family utilize a docking domain known as a DVD (Domain for Versatile Docking) domain that lies outside of the region of phosphorylation on their target substrates. The requirement of the DVD domain for substrate recognition by ASK1has made it a very challenging kinase target.

It is believed that ASK1 activation is associated with elevated cardiac fibrosis, cardiac hypertrophy, dilated cardiomyopathy, increased myocyte apoptosis, and/or loss of functional myocytes. Thus, there is a need for methods for identifying ASK1 inhibitors, and, more particularly, ASK1 inhibitors useful for preventing and/or treating cardiovascular diseases. This invention provides methods that generally address such a need.

Han et al, Bioorganic & Medicinal Chemistry Letters 15 (2005) 5467-5473, discusses identification of coumarin derivatives using a homogenous TR-FRET (time resolved-fluorescence resonance energy transfer)-based in vitro coupling assay that can specifically recognize a phosphorylated ERK substrate peptide.

Douglas Auld, US patent publication No. 2005/0191718, mentions a method of measuring protein phosphatase activity in which a sample is contacted with a reporter peptide which is later subject to treatment of a protease capable of cleaving unphophorylated peptide and then a change in a fluorescence of the reporter peptide is monitored using various methods such as TR-FRET.

### SUMMARY OF THE INVENTION

This invention is directed, in part, to methods for identifying compounds that inhibit ASK1. Such compounds are useful to prevent and/or treat cardiovascular disease in an animal.

This invention also is directed, in part, to methods for preventing and/or treating cardiovascular disease in an animal. The methods comprise administering to the animal an ASK1 inhibitor and, optionally, a hypertensive compound.

Further benefits of Applicants' invention will be apparent to one skilled in the art from reading this patent application.

### BRIEF DESCRIPTION OF THE DRAWINGS

FIGS. 1A and 1B are schematic diagrams of the assay described in Example 1.

FIGS. 2A and 2B are schematic diagrams of the assay described in Example 2.

FIGS. 3A and 3B are schematic diagrams of the assay described in Example 3.

FIG. 4 is a graphical representation of results obtained in the assay described in Example 1.

### DETAILED DESCRIPTION OF THE INVENTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

This invention is directed, in part, to a method for identifying a compound that inhibits ASK1. The method comprises measuring the level of ASK1-dependent phosphorylation of a protein substrate in the absence and presence of the compound, and comparing the level of ASK1-dependent phosphorylation of the protein substrate in the absence and presence of the compound. The compound is useful to prevent and/or treat cardiovascular disease in an animal if the level of ASK1-dependent phosphorylation of the protein substrate in the absence of the compound is higher than the level of ASK1-dependent phosphorylation of the protein substrate in the presence of the compound.

In some embodiments, the compound is a non-protein compound. In some embodiments, the compound is a small organic molecule compound. In some embodiments, the compound has a molecular mass of less than about 1000.

The method of this invention can utilize a variety of protein substrates that can be phosphorylated by ASK1. In some embodiments, the protein substrate comprises a full length physiologically relevant ASK1 substrate, for example, MAP kinase kinase (e.g., MAP kinase kinase 3, MAP kinase kinase 4, MAP kinase kinase 6, or MAP kinase kinase 7). In some embodiments, the protein substrate comprises a generic Ser/Thr substrate, for example, myelin basic protein (MBP). In some embodiments, the protein substrate comprises a fragment of physiologically relevant ASK1 substrate (e.g., a fragment of a MAP kinase kinase). In other embodiments, the protein substrate comprises a fragment of a generic Ser/Thr substrate, for example, a fragment of myelin basic protein. The fragment can be used in the method of the invention as long as it can be phosphorylated by ASK1.

In some embodiments, the protein substrate can be modified to, for example, optimize phosphorylation measurement. In some embodiments, the protein substrate can be biotinylated. In other embodiments, the protein substrate comprises a glutathione S-transferase tag. In other embodiments, the protein substrate comprises a c-myc epitope. In other embodiments, the protein substrate comprises a hemagglutinin epitope. In yet other embodiments, the protein substrate comprises a six histidine tag. In yet other embodiments, the protein substrate comprises troponin T. In further embodiments, the protein substrate comprises a Domain for Versatile Docking.

The level of ASK1-dependent phosphorylation of the protein substrate can be measured in a variety of ways. For example, the measurement can be carried out by employing any assay method suitable to detect phosphorylation of a protein from a biological sample. In some embodiments, the assay method is a fluorescence assay. In some embodiments, the fluorescence assay is TR-FRET. The assay method can be applied to the present invention in a various ways to detect kinase activity of an inhibitor. In some embodiments, phosphorylation is measured directly by monitoring a phosphorylation level of the substrate used. In other embodiments, the level of ASK1-dependent phosphorylation of the protein substrate is measured indirectly (e.g., by utilizing the competitive assay format discussed in Example 2).

This invention is directed, in part, to a method for preventing cardiovascular disease in an animal susceptible to developing cardiovascular disease. Preventing cardiovascular disease includes reducing the risk of, delaying the onset of, and/or keeping an animal from developing cardiovascular disease.

This invention also is directed, in part, to a method for treating cardiovascular disease in an animal in need of such treatment. Treating cardiovascular disease includes ameliorating, suppressing, and/or eradicating cardiovascular disease.

The methods of prevention and treatment of this invention are particularly suitable for use with humans, but may be used with other animals that have kidney(s), particularly mammals, such as, for example, non-human primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

The methods of prevention and treatment of this invention are suitable for a variety of cardiovascular diseases. In some embodiments, the cardiovascular disease comprises diastolic heart failure. In some embodiments, the cardiovascular disease comprises diastolic dysfunction. In some embodiments, the cardiovascular disease comprises cardiac fibrosis. In some embodiments, the cardiovascular disease comprises hypertrophy. In some embodiments, the cardiovascular disease comprises impaired ventricular relaxation. In some embodiments, the cardiovascular disease comprises impaired ventricular filling. In some embodiments, the cardiovascular disease comprises pulmonary artery pressure. In some embodiments, the cardiovascular disease comprises pulmonary hypertension. In some embodiments, the cardiovascular disease comprises pulmonary edema. In some embodiments, the cardiovascular disease comprises shortness of breath. In some embodiments, the cardiovascular disease comprises hypertension. In some embodiments, the cardiovascular disease comprises acute coronary syndrome (including unstable angina and non-Q wave infarction). In some embodiments, the cardiovascular disease comprises myocardial infarction. In some embodiments, the cardiovascular disease comprises heart failure. In some embodiments, the cardiovascular disease comprises systolic heart failure. In some embodiments, the cardiovascular disease comprises stroke. In some embodiments, the cardiovascular disease comprises occlusive stroke. In some embodiments, the cardiovascular disease comprises hemorrhagic stroke.

The methods of treatment and prevention comprise administering to the animal an ASK1 inhibitory compound identified by the screening method discussed above (i.e., the methods comprise administering one or more ASK-1 inhibitors). An ASK1 inhibitor can be administered as a pure compound or a derivative thereof (e.g., a salt, solvate, hydrate, or prodrug of the ASK1 inhibitory compound). Depending on the particular compound, a salt of the compound may be advantageous due to one or more of the salt's physical properties, for example, enhanced pharmaceutical stability in differing temperatures and humidities, or a desirable solubility in water or oil. Preferably the salt is a pharmaceutically-acceptable salt. The term “pharmaceutically-acceptable salt” is used adjectivally to mean that the modified noun is appropriate for use in a pharmaceutical product.

In some embodiments, the methods of prevention and treatment comprise administering to the animal a therapeutically-effective amount of the ASK1 inhibitor. A “therapeutically-effective amount” or “effective amount” means an amount that will achieve the goal of preventing cardiovascular disease (i.e., reducing the risk of, delaying the onset of, and/or keeping an animal from developing cardiovascular disease) in the context of a method of prevention or of treating cardiovascular disease (i.e., ameliorating, suppressing, and/or eradicating cardiovascular disease) in the context of a method of treatment.

In some embodiments, the methods of prevention and treatment further comprise administering to the animal an antihypertensive compound (i.e., the methods of this invention comprise a combination therapy, wherein an ASK1 inhibitor is co-administered with an antihypertensive compound). In some such embodiments, the amount of the ASK1 inhibitor and the amount of the antihypertensive compound together comprise a therapeutically-effective amount.

Antihypertensive drugs suitable for the methods of this invention include, for example, diuretics, angiotensin converting enzyme inhibitors, angiotensin II receptor antagonists, alpha adrenergic receptor antagonists, beta adrenergic receptor antagonists, endothelin receptor antagonists, vasodilators, and calcium channel blockers.

Diuretics suitable for the methods of prevention and treatment of this invention include, for example, acetazolamide, althiazide, ambuside, amiloride, aminometradine, azosemide, bendroflumethiazide, benzthiazide, benzylhydrochlorothiazide, Biogen BG 9719, bumetanide, butazolamide, buthiazide, canrenone, chloraminophenamide, chlorazanil, chlormerodrin, chlorothiazide, chlorthalidone, clofenamide, clopamide, clorexolone, cyclopenthiazide, cyclothiazide, disulfamide, ethacrynic acid, ethiazide, ethoxzolamide, etozolin, fenquizone, furosemide, hydrochlorothiazide, hydroflumethiazide, indapamide, isosorbide, Kiowa Hakko KW 3902, mannitol, mefruside, meralluride, mercaptomerin sodium, mercumatilin sodium, mercurous chloride, mersalyl, methazolamide, methyclothiazide, metolazone, muzolimine, oleandrin, pamabrom, paraflutizide, perhexiline, piretanide, polythiazide, protheobromine, quinethazone, Sanofi-Aventis SR 121463, spironolactone, teclothiazide, theobromine, ticrynafen, torsemide, triamterene, trichlormethiazide, tripamide, urea, and xipamide.

ACE inhibitors suitable for the methods of prevention and treatment of this invention include, for example, alacepril, benazepril, captopril, ceronapril, cilazapril, delapril, enalapril, enalaprilat, eosinopril, fosinopril, imidapril, lisinopril, moexipril, moveltipril, omapatrilat, perindopril, quinapril, ramipril, sampatrilat, spirapril, temocapril, and trandolapril.

Angiotensin II receptor antagonists suitable for the methods of this invention include, for example, candesartan, eprosartan, irbesartan, losartan, olmesartan, tasosartan, telmisartan, and valsartan.

Alpha adrenergic receptor antagonists suitable for the methods of this invention include, for example, doxazosin, phentolamine, phenoxybenzamine, prazosin, terazosin, amosulalol, arotinolol, dapiprazole, ergoloid mesylates, fenspiride, idazoxan, indoramin, labetalol, methyldopa, monatepil, naftopidil, nicergoline, tamsulosin, tolazoline, trimazosin, and yohimbine.

Beta adrenergic receptor antagonists suitable for the methods of this invention include, for example, AC 623, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butidrine hydrocholoride, butofilolol, carazolol, carteolol, carvedilol, celiprolol, cetamolol, cloranolol, dilevalol, esmolol, indenolol, labetalol, landiolol, levobunolol, mepindolol, metipranolol, metoprolol, moprolol, nadolol, nadoxolol, nebivolol, nifenalol, nipradilol, oxprenolol, penbutolol, pindolol, practolol, pronethalol, propranolol, sotalol, sulfinalol, talinolol, tertatolol, tilisolol, timolol, toliprolol, and xibenolol.

Endothelin receptor antagonists suitable for the methods of this invention include, for example, ambrisentan, darusentan, bosentan, avosentan, and TBC3711.

Vasodilators suitable for the methods of this invention include, for example, hydralazine, minoxidil, nicorandil, nitroprusside, amotriphene, benfurodil hemisuccinate, benziodarone, chloracizine, chromonar, cinepazet, clobenfurol, clonitrate, cloricromen, dilazep, droprenilamine, efloxate, erythrityl tetranitrate, etafenone, fendiline, hexestrol bis(β-diethylaminoethyl ether), hexobendine, imolamine, isosorbide dinitrate, isosorbide mononitrate, itramin tosylate, khellin, lidoflazine, mannitol hexanitrate, nitroglycerin, pentaerythritol tetranitrate, pentrinitrol, perhexiline, pimefylline, prenylamine, propatyl nitrate, trapidil, tricromyl, trimetazidine, trolnitrate phosphate, and visnadine.

Calcium channel blockers suitable for the methods of this invention include, for example, amlodipine, aranidipine, bamidipine, bencyclane, benidipine, bepridil, cilnidipine, cinnarizine, clentiazem, diltiazem, dotarizine, efonidipine, elgodipine, etafenone, fantofarone, felodipine, fendiline, flunarizine, gallopamil, isradipine, lacidipine, lercanidipine, lidoflazine, lomerizine, manidipine, mibefradil, monatepil, nicaripine, nifedipine, nilvadipine, nimodipine, nisoldipine, nitrendipine, NZ 105, perhexiline, prenylamine, semotiadil, terodiline, and verapamil.

The preferred total daily dose of the ASK1inhibitor and antihypertensive drug is typically from about 0.001 to about 100 mg/kg, more preferably from about 0.001 to about 30 mg/kg, and even more preferably from about 0.01 to about 10 mg/kg (i.e., mg of agent per kg body weight). It should, however, be recognized that the specific dose level and frequency dosing for each agent will depend on a variety of factors including, for example, the particular combination of agents selected; the activity, efficacy, pharmacokinetic, and toxicology profiles of the particular agents used (including such profiles when the agents are used in combination); the age, weight, general health, sex, and diet of the animal; the frequency of administration; the rate of excretion; the severity of the condition treated; whether a drug delivery system is used; the form, route, and frequency of administration; and whether other pharmaceutically-active compounds are also being administered (to, for example, treat a condition other than cardiovascular disease that the animal suffers from). Thus, the dosage regimen actually employed may vary widely from the dosage regimens set forth in this patent application.

The total daily dose of each therapeutic agent generally may be administered to the animal in a single dose or in proportionate multiple sub-doses. Sub-doses typically are administered from 2 to about 6 times per day, and more typically from 2 to about 4 times per day. Doses may be in an immediate-release form or in a sustained-release form effective to obtain desired results. It should be recognized that, although the dosing frequency for the therapeutic agents in this invention is typically daily or multiple times per day, this invention also contemplates dosing requirements in which the preferred period between administration of one or more of the therapeutic agents is greater than 24 hours. In such embodiments, the dosing frequency may be, for example, every 36 hours, every 48 hours, every 72 hours, weekly, or monthly.

In combination therapies comprising an ASK1 inhibitor and an antihypertensive compound, the administration may comprise administering the ASK1 inhibitor and the antihypertensive compound in a substantially simultaneous manner using either a single formulation (e.g., a single capsule) having a fixed ratio of the therapeutic agents, or separate formulations (e.g., multiple capsules) that each comprise at least one of the therapeutic agents. Such administration also may comprise administering the ASK1 inhibitor and the antihypertensive drug at different times in separate formulations. This may include, for example, administering the components of the combination (e.g., the ASK1 inhibitor and the antihypertensive drug) in a sequential manner; or it may include administering one component multiple times between the administration of another component; or it may include administering two components at the same time, while also separately administering another portion of at least one of those components at a different time as well; or it may include administering the two components sequentially for a two-step effect. Where the components of the combination are dosed separately, the time period between the dosing of each component may range from a few minutes to several hours or days, and will depend on, for example, the properties of each component (e.g., potency, solubility, bioavailability, half-life, and kinetic profile), as well as the condition of the patient.

The therapeutic agents used in the methods of this invention may be administered by any means that produces contact of each agent with its site of action in the body. Each therapeutic agent may each be administered as, for example, a compound per se or a pharmaceutically-acceptable salt thereof. Such salts are often particularly suitable for medical applications because of their greater aqueous solubility relative to the compounds themselves. In some embodiments, all the therapeutic agents are administered orally. In other embodiments, at least one of the therapeutic agents is administered by another means, for example, parenterally.

In some embodiments, a therapeutic agent used in the methods of this invention is administered as part of a pharmaceutical composition (or medicament) that further comprises one or more pharmaceutically-acceptable carriers, diluents, wetting or suspending agents, vehicles, and/or adjuvants (the carriers, diluents, wetting or suspending agents, vehicles, and adjuvants are sometimes being collectively referred to in this patent application as “carrier materials”); and/or other active ingredients. Where the ASK1 inhibitor is administered as part of a combination therapy, the other agent(s) of the combination may also be contained in the same pharmaceutical composition or as a part of a separate pharmaceutical composition or both.

Therapeutic agents (and combinations thereof) suitable for oral administration can be administered in discrete units comprising, for example, solid dosage forms. Such solid dosage forms include, for example, hard or soft capsules, cachets, lozenges, tablets, pills, powders, or granules, each containing a pre-determined amount of the therapeutic agent(s). In such solid dosage forms, the therapeutic agents are ordinarily combined with one or more adjuvants. If administered per os, the therapeutic agents may be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Pharmaceutical compositions particularly suitable for buccal (sub-lingual) administration include, for example, lozenges comprising the therapeutic agent(s) in a flavored base, usually sucrose, and acacia or tragacanth; or pastilles comprising the therapeutic agent(s) in an inert base, such as gelatin and glycerin or sucrose and acacia.

Therapeutic agents (and combinations thereof) suitable for oral administration also can be administered in discrete units comprising, for example, a liquid dosage forms. Such liquid dosage forms include, for example, pharmaceutically acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also may comprise adjuvants, such as wetting, emulsifying, suspending, flavoring (e.g., sweetening), and/or perfuming agents.

Oral delivery of the therapeutic agents in the methods of this invention may include formulations that provide immediate delivery or, alternatively, sustained (or prolonged) delivery of the agent by a variety of mechanisms. Immediate delivery formulations include, for example, oral solutions, oral suspensions, fast-dissolving tablets or capsules, disintegrating tablets, etc. Sustained-delivery formulations include, for example, pH-sensitive release from the dosage form based on the changing pH of the gastrointestinal tract, slow erosion of a tablet or capsule, retention in the stomach based on the physical properties of the formulation, bio-adhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, in the case of capsules, tablets, and pills, the dosage forms may comprise buffering agents, such as sodium citrate, or magnesium or calcium carbonate or bicarbonate. Tablets and pills additionally may be prepared with enteric coatings. Suitable enteric coatings include, for example, cellulose acetate phthalate, polyvinylacetate phthalate, hydroxypropylmethyl-cellulose phthalate, and anionic polymers of methacrylic acid and methacrylic acid methyl ester.

Parenteral administration includes subcutaneous injections, intravenous injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents. Acceptable carrier materials include, for example, water, 1,3-butanediol, Ringer's solution, isotonic sodium chloride solution, bland fixed oils (e.g., synthetic mono- or diglycerides), dextrose, mannitol, fatty acids (e.g., oleic acid), dimethyl acetamide, surfactants (e.g., ionic and non ionic detergents), and/or polyethylene glycols (e.g., PEG 400).

Formulations for parenteral administration may, for example, be prepared from sterile powders or granules having one or more of the carriers materials mentioned for use in the formulations for oral administration. The therapeutic agent(s) may be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

In some embodiments, one or more therapeutic agents are administered via a transdermal device. Other carrier materials and modes of administration known in the pharmaceutical art may also be used.

### EXAMPLES

The following examples are merely illustrative, and not limiting to the remainder of this disclosure in any way.

### Example 1

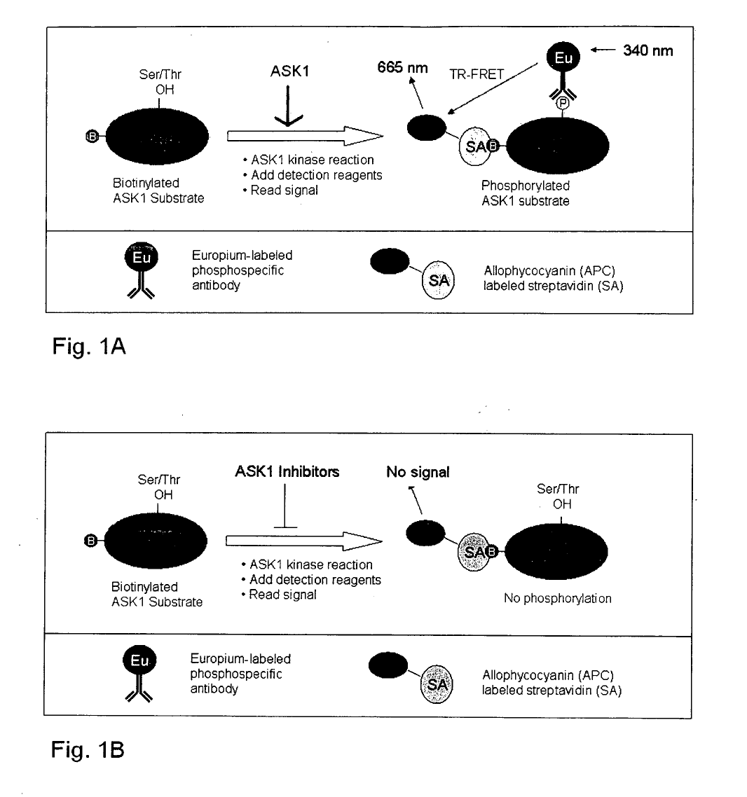

This assay uses biotinylated full length physiologically relevant substrate proteins, such as MAP kinase kinases 3, 6, 4 and 7 (MKK3, MKK6, MKK4 and MKK7) or the generic ser/thr kinase substrate myelin basic protein (MBP). Recombinant ASK1 is used in a kinase reaction to phosphorylate the full length substrate protein. The proximity of the biotin tag and the phosphorylated ser/thr residue on the ASK1 substrate is visualized by bringing two fluorophores in close contact with each other, which allows fluorescence resonance energy transfer (FRET) to occur. One fluorophore (e.g. allophycocyanin) is attached to streptavidin, which binds to the biotin tag, and the other fluorophore (e.g Europium) is attached directly or indirectly to an antibody that recognizes the phosphorylated ser/thr residue. Only those ASK1 substrate molecules that have been phosphorylated will bring the two fluorophores in close enough contact to provide FRET, and thus the resulting assay is a very sensitive measure of ASK1catalytic activity. The use of the lanthanide chelates (e.g. Europium, Terbium, Samarium etc.), which have long fluorescent half lives, creates a TR-FRET based assay that has been shown to be particularly robust for high throughput screening applications. An illustration of this assay is shown in FIGS. 1A and 1B, where the antibody that recognizes the phosphorylation is directly labeled with Europium. An alternative to the TR-FRET based method above is to utilize another homogenous proximity dependent assay format such as AlphaScreen® (PerkinElmer) or scintillation proximity assay (SPA) with the appropriate proximity dependent reagent pairs. In each of these assay formats the inhibition of ASK1 by a small molecule will result in a decreased proximity dependent signal.

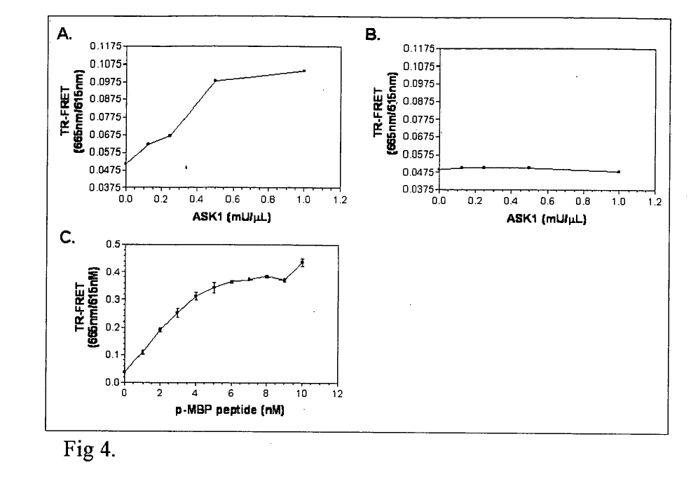

Experimental data using this assay is seen in FIG. 4. In this assay full length biotinylated-MBP (FIG. 4A) and a biotinylated-MBP peptide (90-106) (FIG. 4B) were used as substrates for recombinant constitutively active ASK1. ASK1 was titrated against a constant concentration of either substrate and the ASK1-dependent phosphorylation was monitored using a modification of the detection reagents illustrated in FIG. 1, where the mouse monoclonal antibody that detects MBP phosphorylated at Thr99 is not directly labeled with Europium but a secondary antibody that recognizes the mouse IgG class of antibodies is Europium labeled. This is the indirect method of labeling described for the first assay format. The assay readout is displayed ratiometrically as the TR-FRET dependent APC emission at 665 nm divided by the fluorescence emission of Europium alone when excited with 340 nm light. In FIGS. 4A and 4B, full length biotinylated MBP serves as a substrate for ASK1, whereas the peptide corresponding to the identical phosphorylation site is not a suitable substrate for ASK1. The data in FIG. 4C demonstrates that a synthetic biotinylated-MBP peptide (90-106) containing a phosphate on the Thr99 residue is capable of being recognized by the detection reagents used the assays for FIGS. 4A and 4B, which indicates that the lack of biotin-MBP peptide phosphorylation is due to ASK1 and not the inability of the detection reagents to recognize the phosphorylated Thr99 in the context of a peptide. This allows ASK1 to utilize the full length MBP as a substrate.

### Example 2

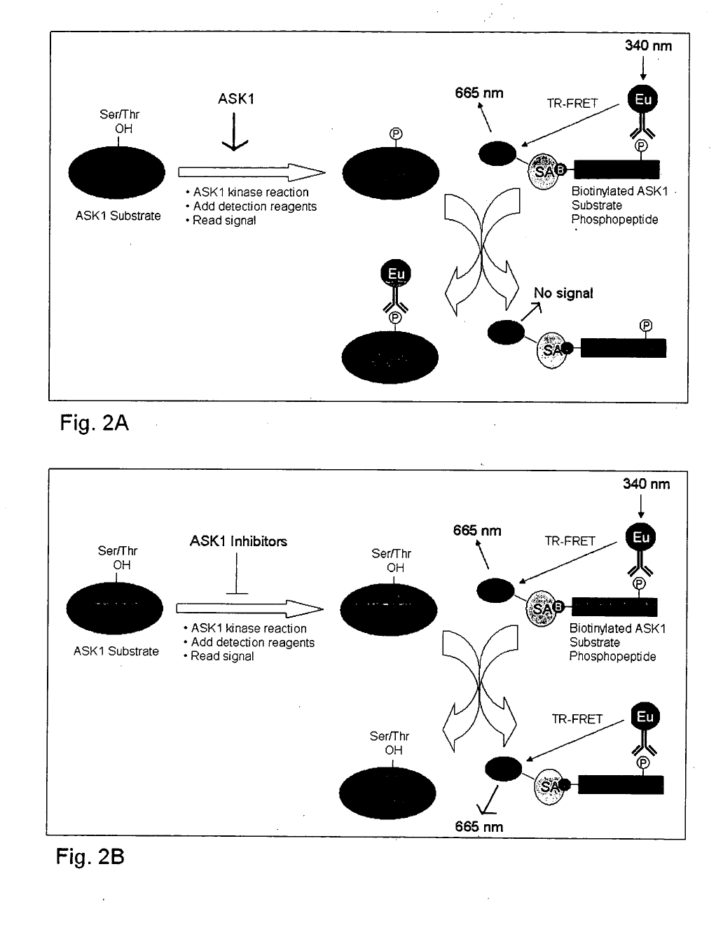

Another format that is suitable for the screening of ASK1 catalytic activity inhibitors is to utilize a competitive assay format. In this assay format, a proximity dependent signal is pre-established on a biotinylated peptide containing a phosphorylated ser/thr residue at the site of ASK1 phosphorylation on the full length substrate. ASK1 is then utilized to phosphorylate an untagged full length substrate producing a phosphorylated full length substrate that competes for the phospho-specific detection reagent that is bound to the peptide. This ultimately leads to a decrease in the proximity dependent signal from the peptide (e.g. TR-FRET, SPA, AlphaScreen®). Consequently, inhibitors of ASK1 will reduce the competition by the full length substrate and result in an increase in the proximity dependent signal from the peptide. An illustration of a TR-FRET version of this assay is shown in FIGS. 2A and 2B, where the antibody that recognizes the phosphorylation is directly labeled with Europium. An alternative format for this assay that would not use proximity dependent reagents is the use of fluorescence polarization (FP). In this format, the peptide would be tagged with a fluorophore instead of biotin and the detection reagent that recognizes the phosphorylated ser/thr residue would add mass to the peptide to create polarization of the fluorescence emission from the peptide. This competitive FP format is analogous to the proximity dependent methods above such that increased ASK1 activity results in a loss of signal (FP) and inhibition of ASK1 results in an increase in assay signal.

### Example 3

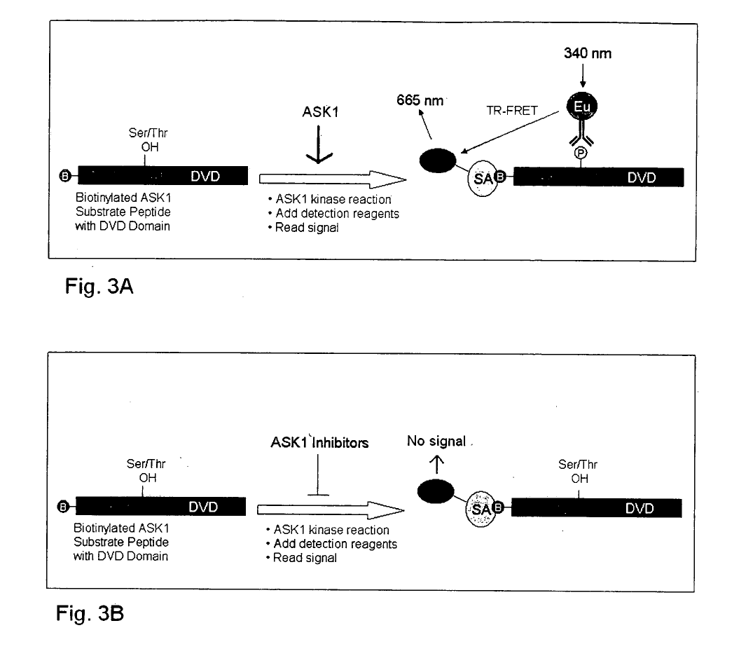

Another assay utilizes peptides derived from ASK1 substrates fused to a DVD docking domain. By providing the necessary docking site, these peptides would act as direct substrates for ASK1. Detection of the ASK1-dependent phosphorylation of these peptide substrates can be performed using proximity based methods (i.e. TR-FRET, AlphaScreen®, SPA) using biotinylated peptides or through the use of FP if the substrate peptide is fluorescently labeled. An illustration of a TR-FRET version of this assay is shown in FIGS. 3A and 3B, where the antibody that recognizes the phosphorylation is directly labeled with Europium. As with the assay described above in Example 1, ASK1 activity causes an increase in the assay readout, whereas inhibition of ASK1 activity is reflected in a loss of assay signal.

### Example 4

This in vivo assay utilizes Dahl SS rats and salt-resistant (SR) rats which are fed a diet containing NaCl at 4-8% of total diet. Increased blood pressure, cardiac hypertrophy and altered LV diastolic function occurs in the SS but not the SR strain after about 5-8 weeks of diet. ASK1 inhibitors are administered to the animals via the IV, IP, SC or oral routes at the initiation of high salt diet or after 5-8 weeks of diet. Duration of treatment is between 2 weeks and 6 months in different experiments.

LV systolic and diastolic functions are measured using a variety of invasive and non-invasive techniques. Serial measurements of LV dimensions, LV function and LV filling are made using echocardiography in animals under light isoflurane anesthesia. The measurements made with this technique include:
LV end systolic diameter
LV end diastolic diameter
LV ejection fraction
LV fractional shortening
LV anterior and posterior wall thicknesses

Using the mitral Doppler flow feature of the echocardiograph, the following measurements of LV filling are made:
early, E-Phase mitral flow velocity
late, A-phase mitral flow velocity
deceleration time of E velocity
isovolumic relaxation time

Under general anesthesia at the termination of each experiment a variety of invasive cardiac and systemic hemodynamics are measured using the high-fidelity, pressure and conductance transducer tipped Millar microcatheter:
Cardiac output (conductance-based measurement)
LV pressure and its first derivative, maximal LV +dP/dt
Systemic blood pressures (systolic, diastolic, pulse and mean blood pressure)
LV relaxation, maximal −dP/dt
LV diastolic properties, LV end-diastolic pressure and volume
LV contractility (LV pressure-volume loop-derived LV contractility index, E_{A}?)
LV relaxation index: tau
Load-dependent systolic and diastolic function, end-systolic pressure volume relation, end-diastolic pressure volume relation

After physiologic measurements have been completed, the hearts are carefully removed and weighed. Cardiac mass is measured and expressed as normalized to body weight, brain weight and tibia length. LV mass is recorded and expressed in a similar fashion. Samples of the LV are emersion-fixed in formalin, sectioned and stained for collagen observation using trichrome stain or Sirius red. Sections are analyzed for collagen area content using a software image analysis system.

Cardiac collagen content and extent of collagen cross-linking is biochemically estimated using the, hydroxyproline assay and determining CNBr-soluble hydroxyproline, respectively.

In some experiments, the hearts are removed and mounted in a Langendorf heart apparatus for determination of LV diastolic compliance properties by determining LV pressure-volume relationships. This procedure is performed prior to sampling the heart for collagen content.

### Example 5

Surgical, coarctation of the aorta (aortic banding) in rats produces immediate and sustained increases in cardiac afterload which results in concentric LV hypertrophy and interstitial cardiac fibrosis. ASK1 inhibitors are administered to the animals via the IV, IP, SC or oral routes at the initiation of pressure-overload or 5-8 weeks of post-operatively. Duration of treatment is between 2 weeks and 6 months in different experiments.

LV systolic and diastolic functions are measured using a variety of invasive and non-invasive techniques. Serial measurements of LV dimensions, LV function and LV filling are made using echocardiography in animals under light isoflurane anesthesia. The measurements made with this technique include:
LV end systolic diameter
LV end diastolic diameter
LV ejection fraction
LV fractional shortening
LV anterior and posterior wall thicknesses

Using the mitral Doppler flow feature of the echocardiograph, the following measurements of LV filling are made:
early, E-Phase mitral flow velocity
late, A-phase mitral flow velocity
deceleration time of E velocity
isovolumic relaxation time

Under general anesthesia at the termination of each experiment a variety of invasive cardiac and systemic hemodynamics are measured using the high-fidelity, pressure and conductance transducer tipped Millar microcatheter:
Cardiac output (conductance-based measurement)
LV pressure and its first derivative, maximal LV +dP/dt
Systemic blood pressures (systolic, diastolic, pulse and mean blood pressure)
LV relaxation, maximal −dP/dt
LV diastolic properties, LV end-diastolic pressure and volume
LV contractility (LV pressure-volume loop-derived LV contractility index, E_{A}?)
LV relaxation index: tau
Load-dependent systolic and diastolic function, end-systolic pressure volume relation, end-diastolic pressure volume relation

After physiologic measurements have been completed, the hearts are carefully removed and weighed. Cardiac mass is measured and expressed as normalized to body weight, brain weight and tibia length. LV mass is recorded and expressed in a similar fashion. Samples of the LV are emersion-fixed in formalin, sectioned and stained for collagen observation using trichrome stain or Sirius red. Sections are analyzed for collagen area content using a software image analysis system.

Cardiac collagen content and extent of collagen cross-linking is biochemically estimated using the hydroxyproline assay and determining CNBr-soluble hydroxyproline, respectively.

In some experiments, the hearts are removed and mounted in a Langendorf heart apparatus for determination of LV diastolic compliance properties by determining LV pressure-volume relationships. This procedure is performed prior to sampling the heart for collagen content.

### Example 6

Rats are fed L-NAME in the drinking water for 5-8 weeks over which period they develop elevated blood pressure and diastolic dysfunction. ASK1 inhibitors are administered to the animals via the IV, IP, SC or oral routes at the initiation of L-NAME administration or after 5-8 weeks of diet. Duration of treatment is between 2 weeks and 6 months in different experiments.

LV systolic and diastolic function is measure using a variety of invasive and non-invasive techniques. Serial measurements of LV dimensions, LV function and LV filling are made using echocardiography in animals under light isoflurane anesthesia. The measurements made with this technique include:
LV end systolic diameter
LV end diastolic diameter
LV ejection fraction
LV fractional shortening
LV anterior and posterior wall thicknesses

Using the mitral Doppler flow feature of the echocardiograph the following measurements of LV filling are made:
early, E-Phase mitral flow velocity
late, A-phase mitral flow velocity
deceleration time of E velocity
isovolumic relaxation time

Under general anesthesia at the termination of each experiment a variety of invasive cardiac and systemic hemodynamics are measured using the high-fidelity, pressure and conductance transducer tipped Millar microcatheter:
Cardiac output (conductance-based measurement)
LV pressure and its first derivative, maximal LV +dP/dt
Systemic blood pressures (systolic, diastolic, pulse and mean blood pressure)
LV relaxation, maximal −dP/dt
LV diastolic properties, LV end-diastolic pressure and volume
LV contractility (LV pressure-volume loop-derived LV contractility index, E_{A})
LV relaxation index: tau
Load-dependent systolic and diastolic function, end-systolic pressure volume relation (ESPVR), end-diastolic pressure volume relation (EDPVR)

After physiologic measurements have been completed, the hearts are carefully removed and weighed. Cardiac mass is measured and expressed as normalized to body weight, brain weight and tibia length. LV mass is recorded and expressed in a similar fashion. Samples of the LV are emersion-fixed in formalin, sectioned and stained for collagen observation using trichrome stain or Sirius red. Sections are analyzed for collagen area content using a software image analysis system.

Cardiac collagen content and extent of collagen cross-linking is biochemically estimated using the hydroxyproline assay and determining CNBr-soluble hydroxyproline, respectively.

In some experiments, the hearts are removed and mounted in a Langendorf heart apparatus for determination of LV diastolic compliance properties by determining LV pressure-volume relationships. This procedure is performed prior to sampling the heart for collagen content.

The terms “comprise”, “comprises”, and “comprising” are to be interpreted inclusively rather than exclusively.

All references (including patent documents) cited above are incorporated by reference into this patent application. The discussion of those references is intended merely to summarize the assertions made by their authors. No admission is made that any of those references (or a portion thereof) is relevant prior art. Applicants reserve the right to challenge the accuracy and pertinence of the cited references.

The above detailed description is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

What is claimed is:

1. A method for identifying an inhibitory compound useful to prevent and/or treat cardiovascular disease in an animal, wherein the method comprises the steps of:
a. measuring the level of ASK1-dependent phosphorylation of a protein substrate in the absence of the inhibitory compound,
b. measuring the level of ASK1-dependent phosphorylation of a protein substance in the presence of the inhibitory compound, and
c. determining the difference in the level of ASK1-dependent phosphorylation of the protein substrate between step a and step b;
wherein the level of ASK1-dependent phosphorylation of the protein substrate in absence of the compound being higher than the level of ASK1-dependent phosphorylation of the protein substrate in presence of the compound, indicates the compound is useful to prevent and/or treat cardiovascular disease in an animal.

2. The method of claim 1, wherein the measuring a phosphorylation level employs a fluorescence assay.

3. The method of claim 2, wherein the fluorescence assay is a TR-FRET assay.

4. The method of claim 1, wherein the compound is a non-protein compound.

5. The method of claim 1, wherein the compound is a small organic molecule compound.

6. The method of claim 1, wherein the compound has a molecular mass of less than about 1000.

7. The method of claim 1, wherein the protein substrate comprises MAP kinase kinase 3 or a portion thereof.

8. The method of claim 1, wherein the protein substrate comprises MAP kinase kinase 4 or a portion thereof.

9. The method of claim 1, wherein the protein substrate comprises MAP kinase kinase 6 or a portion thereof.

10. The method of claim 1, wherein the protein substrate comprises MAP kinase kinase 7 or a portion thereof.

11. The method of claim 1, wherein the protein substrate comprises myelin basic protein or a portion thereof.

12. The method of claim 1, wherein the protein substrate is biotinylated.

13. The method of claim 1, wherein the protein substrate comprises a domain for versatile docking.

14. The method of claim 1, wherein the level of ASK1-dependent phosphorylation of the protein substrate is measured directly.

15. The method of claim 1, wherein the level of ASK1-dependent phosphorylation of the protein substrate is measured indirectly.

16. A method for preventing and/or treating a cardiovascular disease in an animal, wherein the method comprises administering to the animal a compound identified by the method of claim 1.

17. The method of claim 16, wherein the animal is a mammal.

18. The method of claim 16, wherein the animal is a human.

19. The method of claim 16, wherein the cardiovascular disease comprises diastolic heart failure.

20. The method of claim 16, wherein the cardiovascular disease comprises diastolic dysfunction.

21. The method of claim 16, wherein the cardiovascular disease comprises cardiac fibrosis.

22. The method of claim 16, wherein the cardiovascular disease comprises hypertrophy.

23. The method of claim 16, wherein the cardiovascular disease comprises impaired ventricular relaxation.

24. The method of claim 16, wherein the cardiovascular disease comprises impaired ventricular filling.

25. The method of claim 16, wherein the cardiovascular disease comprises elevated pulmonary artery pressure.

26. The method of claim 16, wherein a therapeutically-effective amount of the compound is administered to the animal.

27. The method of claim 16, wherein the method further comprises administering to the animal an antihypertensive compound.
